(19) United States
(12) Patent Application Publication
(10) Pub. No.: US 2012/0143085 A1
(43) Pub. Date: Jun. 7, 2012

(54) TEST ELEMENT EJECTION MECHANISM FOR A METER

(76) Inventors: Matthew Carlyle Sauers, Indianapolis, IN (US); Derek Christopher Lotarski, Noblesville, IN (US)

(21) Appl. No.: 12/958,677

(22) Filed: Dec. 2, 2010

(51) Int. Cl. A61B 5/15

(52) U.S. Cl. 600/573

(57) ABSTRACT

A hand-held biosensing meter includes an ejection mechanism housed by the meter for ejecting a test element from the meter. The meter housing is provided with a port to receive the test element. The ejection mechanism includes a drive portion associated with the receptacle and a trigger portion that is associated with the drive portion and releasably engageable to the housing. Displacement of the trigger portion causes the drive portion to automatically eject the test element from the port of the meter housing. Insertion of a test element into the port re-positions the ejection mechanism to its locked position and provides an indication to the user that the test element is properly inserted into the meter.

### BACKGROUND

As the number of patients suffering from diabetes and similar medical conditions increases, self-monitoring of blood glucose wherein the patient monitors his or her blood glucose levels has become a common practice. The purpose of monitoring the blood glucose level is to determine the concentration level and then to take corrective action, based upon whether the level is too high or too low, to bring the level back within a normal range. The failure to take corrective action can have serious medical implications. Glucose monitoring is a fact of everyday life for diabetic individuals. Failure to test blood glucose levels properly and on a regular basis can result in serious diabetes-related complications, including cardiovascular disease, kidney disease, nerve damage and blindness.

A number of hand-held biosensing meters, such as glucose meters, are currently available that permit an individual to test the glucose level in a small sample of blood. Many of the meter designs currently available make use of a disposable test element which, in combination with the meter measures the amount of glucose in the blood sample electrochemically or optically. When the measurement of the blood sample on the test element is complete, it is necessary to remove the test element from the meter so that the test element can be disposed of and to allow insertion of another test element for a subsequent test. As advancements in test element and meter designs occur, smaller blood sample sizes can be used to measure the amount of glucose in the blood sample. While this allows the size of the test element to decrease, difficulties associated with manually removing smaller test elements from the meter can arise for users of the meter. Furthermore, it is desirable to minimize handling of a used test element to reduce the potential for contamination. Therefore, improvements in the insertion and removal of test elements from meters are desired.

### SUMMARY

A device for ejecting a test element from a meter includes an ejection mechanism inside a housing of the meter. The housing is provided with a port to receive the test element. The ejection mechanism includes a drive portion associated with the port and a trigger portion that is associated with the drive portion and releasably engageable to the housing. Displacement of the trigger portion by the user causes the drive portion to automatically eject the test element from the port of the housing. Insertion of a test element into the opening re-positions the ejection mechanism to its locked position and provides an indication to the user that the test element is properly inserted into the meter.

According to one aspect, a meter comprises an external housing extending around an interior where the housing includes a port that opens between the interior and an exterior of the housing. The meter also includes a connector in the interior of the housing adjacent to the port that is engageable with a test element positioned through the port. The meter also includes an ejection mechanism with a drive portion in the interior of the housing and an arm extending from the drive portion. The ejection mechanism is movable in the housing and relative to the connector between a locked position and a release position. The ejection mechanism is generally biased toward the release position and a portion of the arm positively engages the housing and locks the ejection mechanism in the locked position. The portion of the arm is movable out of positive engagement with the housing to cause at least the drive portion of the ejection mechanism to automatically move from the locked position toward the release position so that the drive portion displaces the test element positioned in engagement with the connector out of the port of the housing.

In one refinement, the meter includes a biasing device engaged to the housing and the ejection mechanism, the biasing device generally biasing the ejection mechanism toward the release position. Exemplary biasing devices include linear springs, coil springs, leaf springs, or cantilevered beam elements, configured and arranged as described further herein.

In another refinement, the drive portion of the ejection mechanism includes a guidance system for guiding movement of the ejection mechanism between the locked and release positions. In one embodiment, the guidance system for the drive portion comprises a central strut and a pair of side rails extending from opposite sides of the central strut. The side rails further extend along opposite sides of the connector so that the connector guides movement of the ejection mechanism between the locked and release positions. Other linear or angular guidance systems may be employed which are independent of the connector, for example opposing guide rails provided in the housing and configured for engagement with the side rails of the drive portion. In a further refinement, the drive portion includes a test element contacting portion projecting outwardly from the central strut between the pair of side rails. The contacting portion extends toward the connector to contact the test element when the test element is positioned in the connector. In yet a further refinement, the arm is resiliently connected to the central strut and extends from one end of the central strut to an outer end of the arm. The arm includes a member that extends from the outer end of the arm, the member being positioned in a hole of the housing when the ejection mechanism is in the locked position. In an additional refinement, the member projects outwardly from the hole of the housing in the locked position and is movable by pressing the member into the hole to deflect the arm to receive the member in the interior of the housing, allowing the biased ejection mechanism to move from the locked position to the release position. The member may also provide an audible indicator when the ejection mechanism moves to the locked position from the release position.

In another refinement, the meter comprises a blood glucose meter with a display and input devices located about the housing, and internal electrical components operatively provided in the interior of the housing.

In a further aspect, a meter system is provided that includes a test element and a meter. The meter includes a meter housing and a port configured to receive at least a portion of a test element for operation with the meter. The meter also includes a connector housing in the interior of the meter housing, generally aligned with and adjacent to the port. The connector housing receives and engages at least a portion of the test element when the test element is inserted into the port. The meter also includes an ejection sled slidably mounted within the interior, such as to the connector housing or guides located within the meter housing. The sled is generally movable within the housing and relative to the connector housing, between a locked position and a release position. The release position is generally located adjacent to the port, and locked position is generally spaced from the release position in a direction away from the port. The ejection sled is biased by a biasing force toward the release position, and is movable to the locked position by insertion of a test element through the port into the connector housing. The sled comprises at least one member that extends into the connector housing, each member having a surface normal to the direction between the release position and locked position, such that a lead edge of the test element engages the member surface to cause displacement of the sled until a locking member provided on the sled releasably engages an anchor point when the sled is in the locked position. When the locking member is disengaged from the anchor point, by user interaction or otherwise, the sled forcibly ejects a test element engaged in the connector housing, wherein the ejection force is generally equal to the biasing force minus any retentional (e.g. frictional) force imparted by the connector housing onto a test element located therein, when the sled is in the locked position. The test element is thereby ejected from the connector housing out of the port in the meter housing.

In a further aspect, a meter comprises an external housing extending around an interior where the housing includes a port that opens between the interior and an exterior of the housing. The meter includes a connector in the interior of the housing adjacent to the port so that the connector is engaged with a test element when the test element is positioned through the port. The meter also includes an ejection mechanism with a drive portion in the interior that is engaged to the connector and a trigger portion connected to the drive portion. The ejection mechanism includes a locked position where the test element is positioned in engagement with the connector and the ejection mechanism is secured in the locked position with the trigger portion. The ejection mechanism is generally biased from the locked position toward a release position where actuation of the trigger portion releases the ejection mechanism from the locked position so that the drive portion contacts the test element in the connector to drive the test element out of the port of the housing.

Further aspects, embodiments, forms, features, benefits, objects, and advantages shall become apparent from the detailed description and figures provided herewith.

### BRIEF DESCRIPTION OF THE FIGURES

FIG. 1 is a perspective view of one embodiment of a meter with a test element ejection mechanism.

FIG. 2 is a cross-section view of a portion of the meter through the test element ejection mechanism.

FIG. 3 is cross-section view of the portion of the meter through the test element ejection mechanism orthogonal to the section view of FIG. 2.

FIG. 4 is a perspective view of one embodiment of the ejection mechanism.

FIG. 5 is a cross section view of the meter with a test element positioned therein and the ejection mechanism in a locked position.

FIG. 6 is a cross section view of the meter with the test element being ejected therefrom with the ejection mechanism moving toward a release position.

FIG. 7 is a schematic view of one embodiment of a biasing mechanism for the test element ejection mechanism.

FIG. 8 is a schematic view of another embodiment of a biasing mechanism for the test element ejection mechanism.

FIG. 9 is a schematic view of yet another embodiment of a biasing mechanism for the test element ejection mechanism.

FIG. 10 is a schematic view of yet another embodiment of a biasing mechanism for the test element ejection mechanism.

FIG. 11 is a perspective view of a portion of a meter housing and an example of a test element ejection mechanism arranged therein.

FIG. 12 is a sectional view of the meter housing and test element ejection mechanism of FIG. 11.

FIG. 13 is a side elevation view of another embodiment of an arrangement between and trigger and sled or driving portion of the ejection mechanism.

FIG. 14 is a plan view of the arrangement shown in FIG. 13.

FIG. 15 is a side elevation view of another embodiment of an arrangement between a trigger and sled or driving portion of the ejection mechanism.

FIG. 16 is a plan view of the arrangement shown in FIG. 15.

### DETAILED DESCRIPTION OF THE ILLUSTRATED EMBODIMENTS

For purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

Various embodiments of the present application are related to an ejection mechanism associated with a hand-held biosensing meter such as a blood glucose meter that allows the user to eject a test element does with a sample fluid from the meter without requiring the user to manually handle or touch the test element. In one embodiment, the user activates a trigger to release the ejection mechanism from a locked position, allowing the ejection mechanism to automatically displace the test element from the meter. In one form, the ejection mechanism is also moved from its released position back to its locked position as another test element is positioned into the meter. The position of the trigger relative to the housing provides an indication that the test element is properly inserted. In one embodiment, the ejection mechanism provides an audible and/or tactile click that communicates to the user that the test element is properly positioned in the meter and ready to measure a sample on the test element.

The ejection mechanism is configured to drive the test element from the meter without requiring the user to supply the force to drive the test element from the housing or requiring gravity to release the test element from the meter. The test element therefore can be retained or alternately ejected while the meter is held in any orientation. In addition, the ejection mechanism is configured so that the test element will not bind in or be caught in the port of the meter as it is ejected, eliminating the need and potential for manual handling of a used test element. The ejection mechanism allows the size of the test element to be reduced since manual handling of the test element is not required to remove it from the meter.

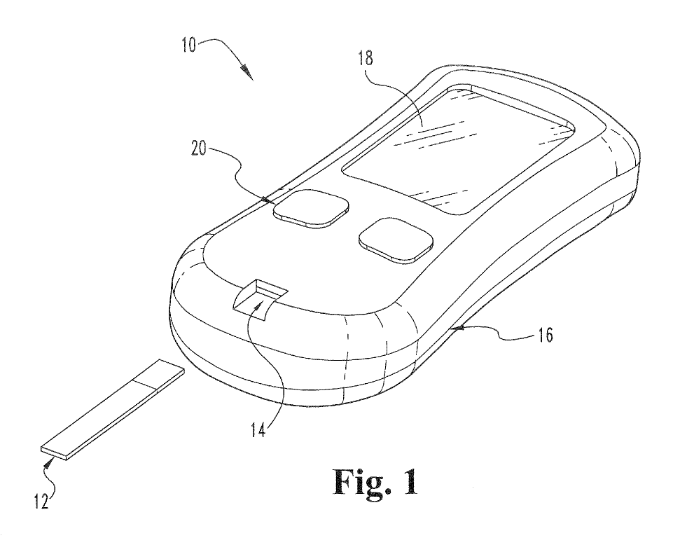

Referring now generally to FIG. 1, there is shown a hand-held biosensing meter 10 and a test element 12 that is positionable into port 14 of meter 10. In one embodiment, meter 10 is a blood glucose meter and test element 12 receives a sample of blood and allows the sample to be tested when the test element is positioned in port 14. In one embodiment, test element 12 is provided as an electrochemical test strip which is useable with a blood glucose meter to perform a blood glucose measurement utilizing electrochemical techniques. In one alternative embodiment, the test element may be provided as a test strip used to perform a blood glucose measurement utilizing optical techniques. An example of a test element configured for use with electrochemical techniques is the ACCU-CHEK® Aviva test strip, which is described more fully in U.S. Pat. No. 7,727,467, the disclosure of which is hereby incorporated herein by reference in its entirety; and an example of a test element configured for use with optical techniques is the ACCU-CHEK® Compact test strip, which is described more fully in U.S. Pat. No. 7,008,799, the disclosure of which is hereby incorporated herein by reference in its entirety. Each of these exemplary test elements are distributed in the United States by Roche Diagnostics Corporation of Indianapolis, Ind. It should be understood, however, that any suitable test element may be employed with the ejection mechanism discussed herein.

Further details and examples of conventional blood glucose meters and related electrical and optical components and their respective measurement techniques are described in U.S. Pat. Nos. 5,352,351; 4,999,482; 5,438,271; 6,645,368; 5,997,817; 6,662,439; RE 36,268; 5,463,467; 5,424,035; 6,055,060; 6,906,802; and 5,889,585; the disclosures of which are hereby incorporated herein by reference in their entireties. It should be understood, however, that the ejection mechanisms discussed herein have application with any suitable meter device that utilizes a test element.

In the illustrated embodiment, meter 10 includes a meter housing 16 that houses a connector housing that releasably connects test element 12 to electrical or optical components of meter 10. Typically, various electrical and computing components are operatively connected to the connector housing in order for the meter to perform the desired analysis of the fluid sample deposited on test element 12. Meter 10 also includes a display 18 to display information to the user, and input devices 20 to allow the user to provide input to the electrical and computing components of meter 10.

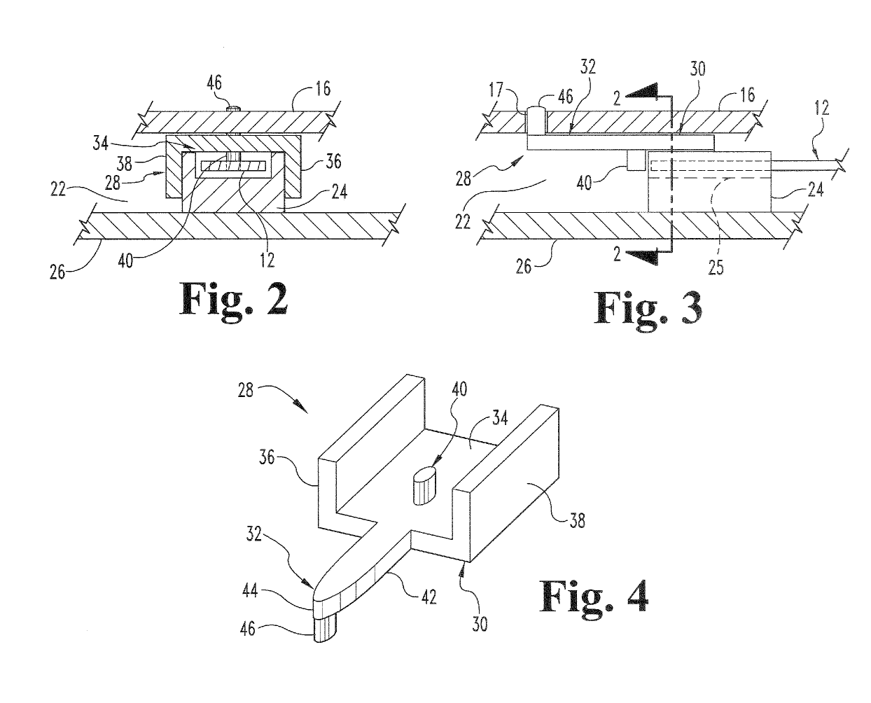

Referring to FIGS. 2-3, there is shown an interior 22 of meter 10 at port 14. Housing 16 extends around interior 22, and housing 16 contains a connector or connector housing 24 that is adjacent to port 14 and connects to test element 12 positioned through port 14. Meter 10 also includes a component 26 adjacent to connector housing 24. In one embodiment, component 26 is a printed circuit board, although other embodiments contemplate that component 26 can be part of connector housing 24, meter housing 16, or other portion of meter 10. Meter 10 also houses ejection mechanism 28 in interior 22 at a location adjacent to connector housing 24. Ejection mechanism 28 includes a sled or drive portion 30 that receives the test element 12 as it is positioned in connector housing 24 and a trigger portion 32 extending from drive portion 30 that is accessible by the user to activate ejection.

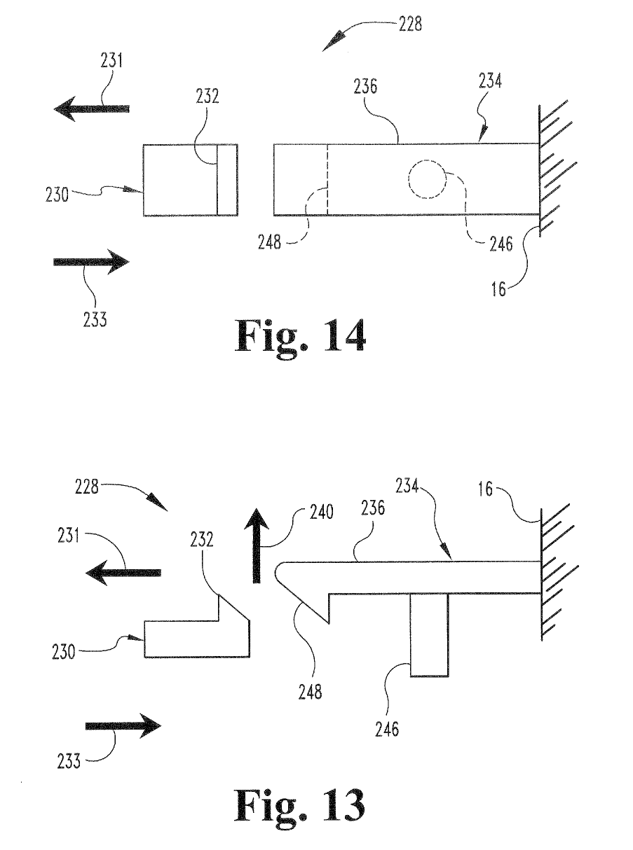

Referring further to FIG. 4, in the illustrated embodiment, drive portion 30 of ejection mechanism 28 includes a central span 34 extending between opposite side rails 36, 38. A test element contacting portion 40 projects outwardly from span 34 between and in the same direction as side rails 36, 38. Contacting portion 40 forms a tab, nub or finger that engages an end of test element 12. Contacting portion 40 provides a surface that extends generally normally to the direction of movement of drive portion 30 between its locked and released positions. In the illustrated embodiment, a single contacting portion 40 is shown. However, other embodiments contemplate two or more contacting portions extending from central span 34. Trigger portion 32 includes an arm 42 extending from an end of span 34 to an outer end 44. Outer end 44 includes a stud, button, knob, lever or other suitable user accessible member 46 projecting therefrom. Member 46 extends from interior 22 and through a hole 17 in housing 16 when ejection mechanism 28 is in its locked position. Hole 17 provides an anchor portion on the housing 16 to which the ejection mechanism is releasably engaged. Hole 17 extends from an interior of the housing to an exterior housing for user access to member 46. Other embodiments contemplate an anchor portion for trigger portion 32 that is located entirely within housing 16, is connected with connector housing 24, such as discussed below with respect to FIGS. 13-16.

In the illustrated embodiment, trigger portion 32 is formed as a unit with drive portion 30. As discussed further below, other embodiments contemplate that the trigger portion is releasably connectable to the drive portion 30 so that the trigger portion 32 locks the drive portion in the locked position, but does not travel with the drive portion when released. In this embodiment, the drive portion 30 re-engages the trigger portion 32 when drive portion is moved to the locked position.

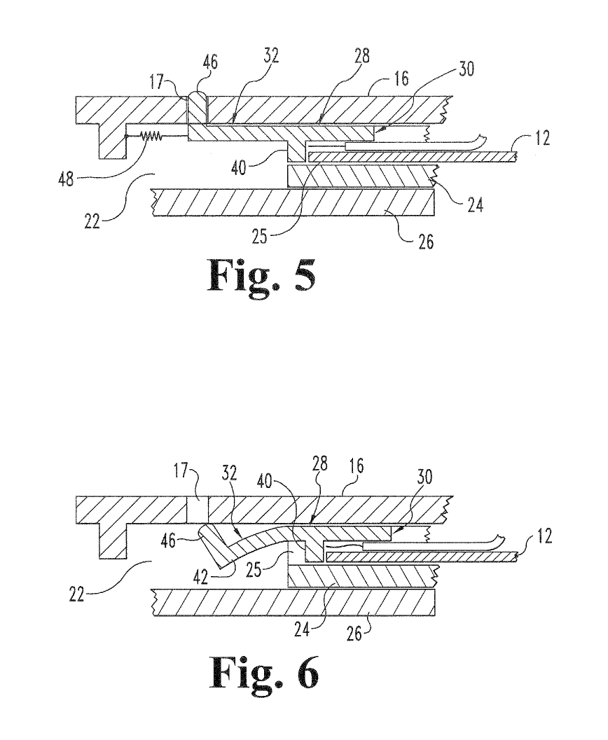

Referring further to FIGS. 5-6, in the illustrated embodiment, ejection mechanism 28 is positioned within housing 16 between connector 24 and housing 16. Side rails 36, 38 extend along opposite sides of connector 24, and connector 24 guides the movement of ejection mechanism 28 between its locked and release positions. Connector 24 and housing 16 constrain movement of drive portion 30 in directions transverse to its path between the locked and release positions. Connector 24 includes at least one slot 25 extending therein that receives contacting portion 40 to contact the end of test element 12 positioned in connector 24 and to allow contacting portion 40 to move along connector 24. A biasing device 48 (FIG. 5) is connected to ejection mechanism 28 and to housing 16 to normally bias ejection mechanism 28 to an unlocked position. In a locked position, such as shown in FIGS. 3 and 5, member 46 projects through hole 17 and positively engages housing 16 where it latches to housing 16 to maintain ejection mechanism 28 in a locked positioned against the bias of biasing device 48. When it is desired to eject test element 12 from port 14, the user presses member 46 into housing 16, which in turn bends arm 42 from its normal locked orientation to unlatch member 46 from housing 16. This allows the bias of biasing device 48 to move ejection mechanism 28 toward the opening of port 14 into housing 16, as shown in FIG. 6, until ejection mechanism 28 rests in a release position. During this movement, contacting portion 40 contacts the end of test element 12 and moves test element 12 along connector 24 as ejection mechanism 28 moves along connector 24. The travel of ejection mechanism 28 in housing 16 is sufficient to move test element 12 along connector 24 and through the opening of port 14 so that test element 12 is ejected from housing 16 for suitable disposal.

Once test element 12 is ejected from housing 16, another test element 12 can be inserted into port 14 while ejection mechanism 28 is in its release position. As the test element 12 is inserted, the leading end of the test element 12 contacts contacting portion 40 of ejection mechanism 28 and drives ejection mechanism 28 toward its locked position and against the bias of biasing device 48. When member 46 aligns with hole 17 of housing 16, arm 42 returns towards its pre-bent configuration and member 46 is received in hole 17 when the locked position is obtained. In this position, test element 12 is properly positioned relative to connector 24 to obtain a sample reading. When member 46 protrudes through hole 17, it provides the user a visual indication that test element 12 is properly inserted through port 14 and into connector 24. In a further embodiment, engagement of member 46 with the hole 17 of meter housing 16 may configured to provide an audible click when moving into the locked position, providing a further indication to the user that the test element 12 is properly positioned relative to connector 24, as shown in FIG. 5.

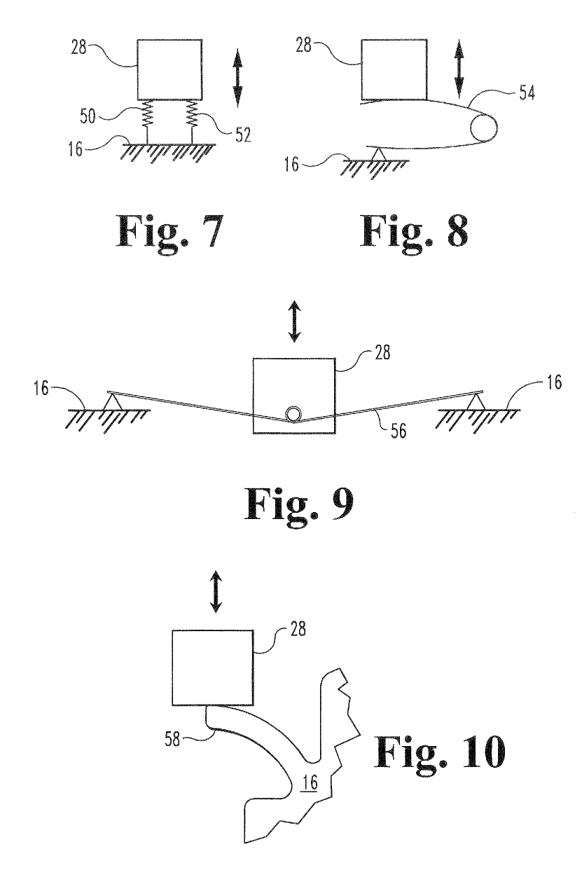

FIGS. 7 and 8 show alternate embodiment biasing devices 48. In FIG. 7, biasing device 48 includes a pair of linear springs 50, 52 that are connected to housing 16 and ejection mechanism 28. Springs 50, 52 are compressed (or distended) when ejection mechanism 28 is locked and are configured to displace ejection mechanism 28 toward the release position when ejection mechanism 28 is released with activation of 46. In FIG. 8, an angular spring 54 is connected to housing 16 and ejection mechanism 28. Spring 54 compresses when ejection mechanism 28 is locked and is configured to displace ejection mechanism 28 toward the release position when ejection mechanism 28 is released with activation of 46. Other embodiments contemplate other forms for the biasing device, including a single linear spring, more than two linear springs, multiple angular springs, arch springs, leaf springs, coil springs, torsion springs, washer springs, and elastic material, for example. FIGS. 9 and 10 provide illustrative embodiments of certain of these alternative biasing devices. FIG. 9 shows a coil or leaf spring 56 connected to housing 16 or other part of meter 10 that biases ejection mechanism 28. FIG. 10 shows a cantilevered resiliently deformable beam 58 connected to housing 16 or other part of meter 10 that contacts and biases ejections mechanism 28.

The biasing device 48 supplies translation energy by pushing or pulling ejection mechanism 28 from the locked position to the release position. The biasing force is sufficient to overcome the frictional forces that tend to retain ejection mechanism 28 in position relative to housing 16 and connector 24. These forces include the frictional force of the test element 12 against contacts of connector 24 and the frictional force of ejection mechanism 28 against housing 16 and connector 24. Furthermore, the biasing force supplied by biasing device 48 is not so great as to prevent the user from moving ejection mechanism 28 against the biasing force from its release position to its locked position via insertion of the test element into port 14. In addition, member 46 engages housing 16 with sufficient force to prevent the biasing device 48 from moving ejection mechanism 28 in housing 16 until member 46 is unlatched or activated by the user to disengage it from housing 16.

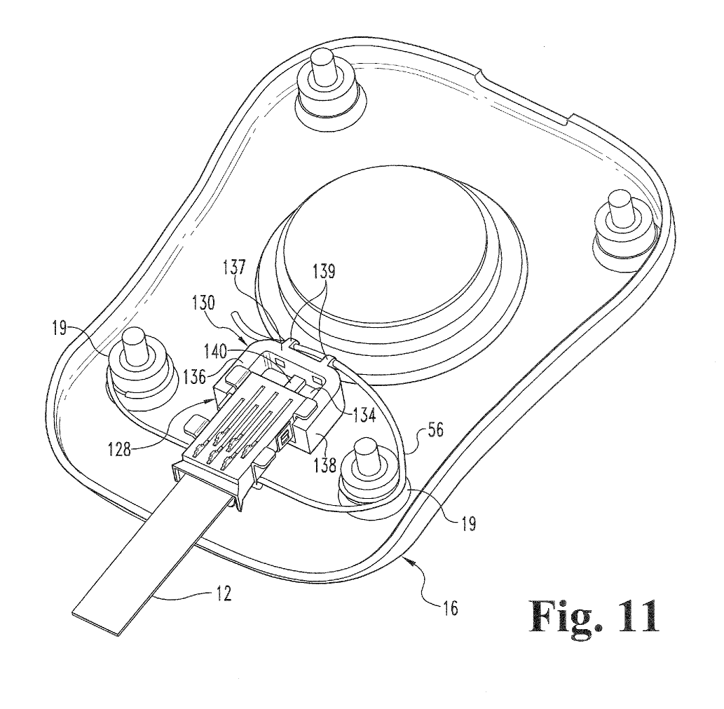

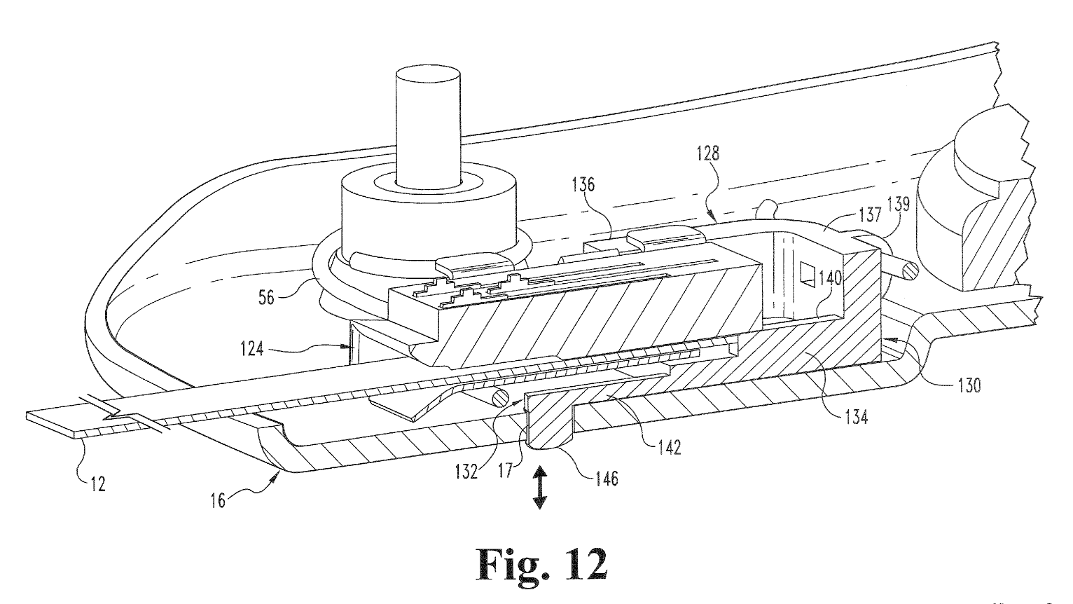

Referring to FIGS. 11-12, there is shown a portion of housing 16 for a meter with an embodiment of an ejection mechanism 128 connected with a biasing device 56 in the form of a coil spring. Housing 16 houses a connector housing 124 that is adjacent to a port in housing 16, such as port 14 discussed above. Connector housing 124 connects to test element 12 positioned through the port 14. Meter housing 16 also houses ejection mechanism 128 in its interior at a location adjacent to connector housing 124. Ejection mechanism 128 includes a sled or drive portion 130 that receives at least a portion of connector housing 124 and a trigger portion 132 extending from drive portion 130 along connector housing 124. Trigger portion 132 is accessible by the user to activate ejection mechanism 128. Ejection mechanism 128 is similar to ejection mechanism 28 discussed above except for the direction in which trigger portion 132 extends from drive portion 130. Of course, it is to be understood that trigger portion 132 could be arranged to extend away from drive portion 130 in a direction opposite that shown in FIGS. 11-12, or arranged extend in some intermediate direction.

Drive portion 130 of ejection mechanism 128 includes a central strut 134 extending between opposite side rails 136, 138. A test element contacting portion 140 projects outwardly from strut 134 between and in the same direction as side rails 136, 138. Contacting portion 140 forms a nub or finger that extends generally normal to the direction of movement of drive portion 130 and engages an end of test element 12. Trigger portion 132 includes an arm 142 extending from an end of strut 134 to an outer end having a button, knob, lever or other suitable user accessible member 146 projecting therefrom. Member 146 extends from the interior of meter housing 16 and through a hole 17 in housing 16 when ejection mechanism 128 is in its locked position, as shown in FIGS. 11 and 12. Ejection mechanism 128 is positioned within meter housing 16 between connector housing 124 and meter housing 16. Side rails 136, 138 extend along opposite sides of connector housing 124, and connector housing 124 guides the movement of ejection mechanism 128 between its locked and released positions. Connector housing 124 and meter housing 16 constrain movement of drive portion 130 in directions transverse to its path between the locked and release positions. Connector housing 124 is configured to receive contacting portion 140 to contact the end of test element 12 positioned in connector housing 124 and to allow contacting portion 140 to move along connector housing 124 to eject the test element 12.

Biasing device 56 is connected to ejection mechanism 128 and to meter housing 16 to bias ejection mechanism 128 toward an unlocked position. In a locked position, such as shown in FIGS. 11-12, member 146 projects through hole 17 and positively engages meter housing 16 where it latches to meter housing 16 to maintain ejection mechanism 128 in a locked positioned against the bias of biasing device 56. In the illustrated embodiment, biasing device 56 is in the form of a coil spring that is secured around posts 19 that are connected with meter housing 16 on opposite sides of connector housing 124. The free end of the coil spring extends through ears 139 projecting outwardly from an endwall 137 of driving portion 130. The coil spring pushes on drive portion 130 to bias ejection mechanism 128 away from its locked position shown in FIGS. 11-12 toward a release position that ejects test element 12 from port 14.

When it is desired to eject test element 12 from port 14, the user presses member 146 into meter housing 16, which in turn bends arm 142 from its normal locked orientation to unlatch member 146 from meter housing 16. This allows the bias of biasing device 56 to move ejection mechanism 128 toward the opening of port 14 of meter housing 16 until ejection mechanism 128 obtains a release position. During this movement, contacting portion 140 contacts the end of test element 12 and moves test element 12 along connector housing 124 as ejection mechanism 128 moves along connector housing 124. Once test element 12 is ejected from housing 16, another test element 12 can be inserted into port 14 while ejection mechanism 128 is in its release position. As the test element 12 is inserted, the leading end of the test element 12 contacts contacting portion 140 of ejection mechanism 128 and drives ejection mechanism 128 toward its locked position and against the bias of biasing device 56.

FIGS. 13-14 show another embodiment arrangement between the trigger and sled or driving portion of the ejection mechanism. In particular, the trigger portion is separate from the sled portion of the ejection mechanism and releasably engages the sled portion when the sled portion is in its locked position. As shown in FIGS. 13-14, ejection mechanism 228 includes a drive or sled portion 230 that includes a ledge 232 projecting therefrom. The trigger portion 234 is secured to housing 16 or other feature in the device, such as an internal frame and includes a beam portion 236 extending from housing 16. Beam portion 236 includes a button or other suitable release member 246 projecting from a side of beam portion 236. Member 246 extends through hole 17 or other suitable location or mechanism associated with the housing 16 so that it is accessible by the user. Beam portion 236 also includes a catch ledge 248 at one end thereof that provides an anchor portion in housing 16 to engage ledge 232 when sled portion 230 is in the locked position. In order to release sled portion 230, the user presses or otherwise manipulates member 246 to bend or deflect beam portion 236 as indicated by arrow 240 until catch ledge 248 no longer contacts sled ledge 232. The biasing device is mounted to the sled portion 230 so that sled portion 230 then moves, as indicated by arrow 231, toward the release position where it contacts and ejects a test element as discussed above. Insertion of a test element in port 14 moves sled portion 230 against the bias of the biasing device, as indicated by arrow 233, until sled ledge 232 is positioned beneath catch ledge 248. The end surfaces of sled portion 230 and beam portion 236 can be sloped, chamfered, or otherwise configured to facilitate bending of beam portion 236 to allow passage of sledge ledge 232 along the projecting part of catch ledge 248. Furthermore, beam portion 236 can be made from a flexible material, such as plastic or thin metal material, that allows bending of beam portion 236 and provides resiliency for beam portion to return to or toward its pre-bent position when the bending force is released.

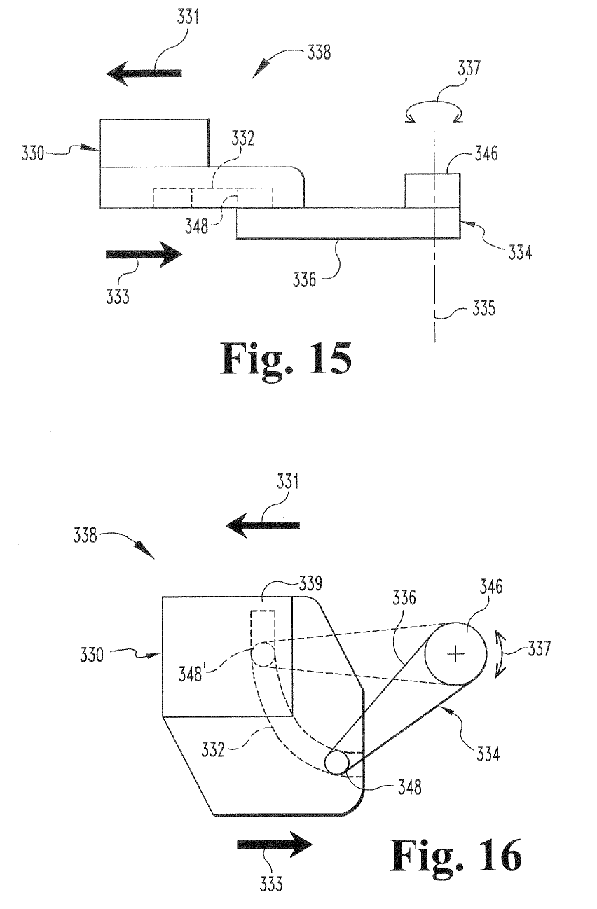

FIGS. 15-16 show another embodiment arrangement between the trigger and sled or driving portion of the ejection mechanism where the trigger portion is separate from the sled portion of the ejection mechanism. Ejection mechanism 328 includes a driving or sled portion 330 that includes a catch track 332 defined thereby along a curved path. The trigger portion 334 is rotatably secured to housing 16 and includes an arm portion 336 extending from housing 16. Arm portion 336 includes a member 346 projecting from a side of arm portion 336 on an axis 335 about which trigger portion 334 rotates. Member 346 can extend through housing 16, or otherwise catch on a feature that can be part of the sled portion 330 or trigger portion 334. The opposite end of arm portion 336 includes a pin or catch 348 that is positioned in catch track 332.

When trigger portion 334 is in its unlocked position as shown in FIGS. 15-16, catch 348 is aligned with a portion of catch track 332 that allows sled portion 330 to move under bias from the biasing device in a direction indicated by arrow 331 that ejects the test element. When the next test element is inserted into the connector housing, it contacts sled portion 330 and moves it toward trigger portion 334. As catch 348 moves along catch track 332, arm portion 336 rotates about axis 335, as indicated by arrow 337, to position catch 348 in a portion of catch track 334 that extends transverse to the direction of movement 331, 333 of sled portion 330, as indicated by the positioning of catch 348′. In this position, trigger portion 334 restrains sled portion 330 from movement caused by the biasing device until trigger portion 334 is rotated about axis 335 to align catch 348 in the portion of catch track 332 that parallels the direction of movement 331, 333 of sled portion 330. Sled portion 330 can include a stop 339 in catch track 332 to prevent over-rotation of trigger portion 334. It is contemplated that the biasing device can be engaged to sled portion 330, trigger portion 334, or both. In addition, the biasing device can be a linear or angular spring, depending on the mounting of ejection mechanism 328 on housing 16. Trigger portion 334 can be manually activated or engaged by any suitable knob, switch, button slide or other device that allows sled portion 330 to be released when it is desired to eject the test element.

While the invention has been illustrated and described in detail in the drawings and foregoing description, the same is to be considered as illustrative and not restrictive in character, it being understood that only certain embodiments have been shown and described and that all changes and modifications that come within the spirit of the inventions are desired to be protected. It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

What is claimed is:

1. A meter, comprising:
an external housing extending around an interior, said housing including a port that opens between said interior and an exterior of said housing;
a connector in said interior of said housing adjacent to said port, said connector being engageable with a test element positioned through said port; and
an ejection mechanism including a drive portion in said interior and an arm extending from said drive portion, said ejection mechanism being movable in said housing and relative to said connector between a locked position and a release position, wherein said ejection mechanism is biased toward said release position and a portion of said arm positively engages said housing and locks said ejection mechanism in said locked position, said portion of said arm being movable out of said positive engagement with said housing to cause at least said drive portion of said ejection mechanism to automatically move from said locked position toward said release position so that said drive portion displaces the test element positioned in engagement with said connector out of said port of said housing.

2. The meter of claim 1, further comprising a biasing device engaged to said housing and said ejection mechanism that normally biases said ejection mechanism toward said release position.

3. The meter of claim 2, wherein said biasing device is selected from the group consisting of at least one linear spring and at least one angular spring.

4. The meter of claim 1, wherein said drive portion includes a central strut and a pair of side rails extending from opposite sides of said central strut, said side rails further extending along opposite sides of said connector so that said connector guides movement of said ejection mechanism between said locked and release positions.

5. The meter of claim 4, wherein said drive portion includes a test element contacting member projecting outwardly from said central strut between said pair of side rails, said contacting element extending toward said connector to contact the test element when positioned in said connector.

6. The meter of claim 5, wherein said arm is resiliently connected to said central strut and extends from one end of said central strut to an outer end of said arm, said arm including a member that extends from said outer end of said arm that is positioned in a hole of said housing when said ejection mechanism is in said locked position.

7. The meter of claim 6, wherein said member projects outwardly from said hole of said housing in said locked position and is movable by pressing said member into said hole to deflect said arm to receive said member in said interior, thereby allowing said biased ejection mechanism to move from said locked position to said release position.

8. The meter of claim 7, wherein said member extending from said end of said arm provides an audible indicator when said ejection mechanism moves to said locked position from said release position.

9. The meter of claim 1, wherein said external housing comprises a blood glucose meter with a display, input devices and internal electrical components in said interior.

10. A meter, comprising:
a meter housing having a port configured to receive a test element;
a connector housing provided in an interior of the meter housing, the connector housing being generally aligned with the port and configured to receive and engage a portion of a test element inserted into the port;
an ejection sled slidably mounted within the meter housing and movable relative to the connector housing between a release position and a locked position, the release position being located generally adjacent to the port, the locked position being spaced from the release position away from the port, the sled comprising at least one member extending into the connector housing, each member having a surface oriented generally normal to the direction between the locked and release positions, the sled further comprising a locking member configured to releasably engage an anchor portion within the meter housing when the sled is moved into the locking position, wherein the sled is biased by a biasing force into the release position, the sled being movable to the locked position by insertion of a test element through the port into the connector housing wherein a leading edge of such test element engages the normal surface to cause displacement of the sled until the locking member engages the anchor portion, and wherein the sled is configured to eject a test element when the locking member disengages from the anchor portion with an ejection force generally equal to the biasing force minus any retentional force imparted by the connector housing onto a test element located therein when the sled is in the locked position.

11. The meter of claim 10, wherein the anchor portion is located on the connector housing.

12. The meter of claim 10, wherein the anchor portion is provided on an interior surface of the meter housing.

13. The meter of claim 10, wherein the sled is slidably mounted to the connector housing.

14. The meter of claim 10, wherein the sled is slidably mounted to an interior surface of the meter housing.

15. A meter, comprising:
a meter including an external housing extending around an interior, said housing including a port that opens between said interior and an exterior of said housing;
a connector in said interior of said housing adjacent to said port, said connector being engaged with a test element when said test element is positioned through said port; and
an ejection mechanism including a drive portion in said interior that is engaged to said connector and a trigger portion associated with said drive portion, said ejection mechanism including a locked position where the test element is positioned in engagement with said connector and said ejection mechanism is secured in said locked position with said trigger portion, said ejection mechanism being generally biased from said locked position toward a release position where actuation of said trigger portion releases said ejection mechanism from said locked position so that said drive portion contacts the test element in said connector to drive the test element out of said port of said housing.

16. The meter of claim 15, wherein:
said trigger portion includes an arm that is resiliently connected to said central strut and said arm extends from said central strut to an outer end of said arm, said arm including a member extending from said outer end that is positioned in a hole of said housing when said ejection mechanism is in said locked position; and
said member projects outwardly from said hole in said locked position and is movable by pressing said member into said hole to deflect said arm to receive said member in said interior, thereby allowing said biased ejection mechanism to move from said locked position to said release position.

17. The meter of claim 15, wherein said drive portion includes:
a central strut and a pair of side rails extending from opposite sides of said central strut, said side rails extending along opposite sides of said connector so that said connector guides movement of said ejection mechanism between said locked and release positions; and
a test element contacting member projecting outwardly from said central strut between said pair of side rails, wherein said test element contacting member is received in a slot of said connector to contact an end of the test element positioned in said connector.

18. The meter of claim 15, further comprising a biasing device engaged to said housing and said ejection mechanism, said biasing device normally biasing said ejection mechanism toward said release position.

19. The meter of claim 15, wherein said trigger portion releasably engages said drive portion of said ejection mechanism.

20. The meter of claim 19, wherein:
said trigger portion includes a beam portion connected to said housing and a catch ledge extending from said beam portion;
said drive portion includes a ledge extending therefrom that releasably engages said catch ledge of said trigger portion when said ejection mechanism is in said locked position; and
said beam portion is bendable to release said catch ledge from said ledge of said drive portion to allow said drive portion to move under said bias from said locked position toward said release position.

21. The meter of claim 19, wherein:
said trigger portion includes an arm portion rotatably attached to said housing and a catch extending from said arm portion; and
said drive portion includes a catch track in which said catch is slidably and rotatably received, said catch track extending from a first location where said catch engages said drive portion in said catch track to maintain said drive portion in said locked position and said arm portion is rotatable about an axis to move said catch along said catch track to position said catch in a second location of said catch track where said drive portion is allowed to move under said bias from said locked position toward said release position.
